(19) United States
(12) Patent Application Publication
(10) Pub. No.: US 2026/0209716 A1
(43) Pub. Date: Jul. 23, 2026

(54) CELL EXTRACTION METHOD, CELL EXTRACTION SYSTEM, AND SUBCULTURE METHOD

(71) Applicants: CANON KABUSHIKI KAISHA, Tokyo (JP); CANON MEDICAL SYSTEMS CORPORATION, Tochigi (JP)

(72) Inventors: YUHI HIGUCHI, Tokyo (JP); YUSUKE OWAKI, Tochigi (JP)

(21) Appl. No.: 19/455,143

(22) Filed: Jan. 21, 2026

(30) Foreign Application Priority Data
Jan. 22, 2025 (JP) 2025-009481

(51) Int. Cl. C12N 5/074; C12M 1/00; C12M 1/26

(52) U.S. Cl. CPC C12N 5/0696; C12M 29/06; C12M 33/04; C12N 2509/00

(57) ABSTRACT

A cell extraction system includes a first detachment mechanism, a second detachment mechanism, and a collection mechanism. The first detachment mechanism is configured to detach at least a part of a first cell from a culture vessel, wherein contents of the culture vessel comprise the first cell and a second cell which has a higher adhesion strength than the first cell which coexist in the culture vessel, by applying a first physical force to the contents of the culture vessel via a first liquid after the first and second cells are immersed in the first liquid containing a detachment reagent. The second detachment mechanism is configured to detach the second cell present in the culture vessel by applying a second physical force larger than the first physical force to the content of the culture vessel via a second liquid injected into the culture vessel after the first liquid containing the first cell detached is discharged from the culture vessel.

### BACKGROUND

### Field of the Technology

The present disclosure relates to a cell extraction method, a cell extraction system, and the like for selectively detaching and extracting a cell from inside a vessel.

### Description of the Related Art

Induced pluripotent stem (iPS) cells are pluripotent stem cells having the ability to differentiate into various types of cells of tissues and organs and the ability to proliferate almost indefinitely. By introducing a reprogramming factor into a mononuclear cell, reprogramming of genetic information of a cell is started. By culturing the cell in a dedicated culture medium for several days, cell reprogramming is completed, and the iPS cells can be established. A known method for introducing the reprogramming factor uses a Sendai virus vector.

To establish the iPS cells, first, the mononuclear cell is infected with a viral vector. The cell infected with the viral vector starts to be reprogrammed and is changed from a suspension cell to an adherent cell. The cell that has been changed to the adherent cell proliferates on a bottom surface of a culture vessel, and the cell that has completed reprogramming becomes the iPS cell and proliferates while forming a colony. However, the partially reprogrammed adherent cell also proliferates and grows close to the iPS cell colony. Therefore, both the iPS cells and the partially reprogrammed cells containing the reprogramming factor coexist in the culture vessel.

The partially reprogrammed cell does not exhibit pluripotency and may thus hinder differentiation induction. In addition, the partially reprogrammed cell that does not differentiate is an unnecessary cell to be removed before differentiation induction, because the partially reprogrammed cell has the potential to form tumors in vivo.

The partially reprogrammed cell can be identified using green fluorescent protein (GFP). By using a viral vector programmed to produce the GFP within the cell, the cell that has been infected and has started to be reprogrammed emits green fluorescence. Once the reprogramming is completed and the cell becomes the iPS cell, the cell no longer emits green fluorescence. In contrast, the partially reprogrammed cell continuously emits green fluorescence because a gene derived from the reprogramming factor remains in the cell. Accordingly, the cell expressing the GFP is the partially reprogrammed cell to be removed. In other methods, the partially reprogrammed cell can also be identified by using a surface antigen marker. Examples of the surface antigen marker include an SSEA-1 surface antigen marker. A cell that is positive for SSEA-1 is a cell that does not exhibit pluripotency like the iPS cell does. Therefore, similar to the case of using the GFP, it is possible to identify the partially reprogrammed cell.

Previously, the partially reprogrammed cell has been removed by performing manual colony picking. By detaching and collecting a cell positioned at the center of the iPS cell colony manually with a manipulator or the like and subculturing the cell, only the iPS cell can be proliferated. However, colony picking may cause contamination with the partially reprogrammed cell, contamination of cells by microorganisms, damage of cells due to mechanical stimulation (damage), and the like, depending on the skill level of an operator. For this reason, it is necessary for a skilled technician to carefully perform detachment work while securing and maintaining a sterile environment, which has made such work practically difficult.

JP 2017-201946 A describes a cell culture device that co-cultures a human iPS cell with a cell that is not derived from human, such as a mouse fibroblast, as a feeder cell, and separately collects the respective cells. Specifically, JP 2017-201946 A discloses that the cell culture device is a device in which a first rocking motion is applied to a culture vessel and then a second rocking motion is applied in order to detach the cell from the culture vessel, and the first rocking motion has a greater number of rocking cycles, a higher rocking frequency, and a larger rocking angle than the second rocking motion.

JP 2014-18185 A discloses a culture method and a subculture method for applying high-frequency vibration to a culture vessel to detach a cell from a cell adhesion surface in a region-selective manner.

The device disclosed in JP 2017-201946 A is considered to be effective to some extent in a system in which the cell that is not derived from human, such as a mouse fibroblast, is co-cultured as the feeder cell, but the inventors of the present disclosure have found that there are the following problems.

For example, in the case of producing the iPS cell from a mononuclear cell as described above, it has been difficult with the method described in JP 2017-201946 A to separate and collect the iPS cell and the partially reprogrammed cell at a high yield. This is because in a case where the iPS cell is produced from a mononuclear cell, a difference in adhesion strength between two types of cells to be separated, that is, the iPS cell and the partially reprogrammed cell, is small. For this reason, as disclosed in JP 2017-201946 A, when a large rocking motion is applied to the culture vessel at the beginning, both the iPS cell and the partially reprogrammed cell may be detached, and the iPS cell and the partially reprogrammed cell may be collected together without being separated.

In addition, in JP 2017-201946 A, in order to control adhesion strengths of the human iPS cell and the mouse fibroblast cell serving as the feeder cell, an immersion time in a detachment reagent and the number of incubations are controlled. Therefore, the iPS cell is immersed in the detachment reagent for a long time or a plurality of times, as a result of which protein on the surface of the iPS cell may be denatured by the detachment reagent, leading to deterioration of the quality of the cell. For example, adhesiveness of the iPS cell to the culture vessel used for subculture may decrease, resulting in a problem that subculture cannot be performed.

The method described in JP 2014-18185 A is a method of physically selecting a region in the culture vessel, applying a detaching force, and detaching the cell. Therefore, it is considered that there is a possibility that the cells can be selectively detached in a case where cells to be separated are non-uniformly distributed in the culture vessel in each region that is wide to a certain extent in a distinguishable manner, but the inventors of the present disclosure have found that there are the following problems.

For example, in a case where the iPS cell and the partially reprogrammed cell coexist in the culture vessel, it is not easy to selectively detach the cell. Even if local regions are sequentially selected and detached, a processing time from immersion in the detachment reagent to completion of detachment and subculture becomes long, which may cause deterioration in the quality of the iPS cell. For example, adhesiveness of the iPS cell to the culture vessel used for subculture may decrease, resulting in a problem that subculture cannot be performed.

Therefore, there has been a demand for a technology advantageous for distinguishing cells having a small difference in adhesion strength, which are present in a vessel, and extracting a target cell at a high selection ratio.

### SUMMARY

According to a first aspect of the present disclosure, a cell extraction method includes a detachment reagent injection step, followed by a first detachment step, a second detachment step, and a collection step. The detachment reagent injection step is a step of injecting a first liquid containing a detachment reagent into a culture vessel in which a first cell and a second cell coexist to make an adhesion strength of the first cell lower than an adhesion strength of the second cell. The first detachment step additionally includes applying, by a first detachment mechanism, a first physical force to a content of the culture vessel via the first liquid to detach at least a part of the first cell and discharging from the culture vessel the first liquid containing the first cell detached. The second detachment step is a step of supplying a second liquid to the culture vessel and applying, by a second detachment mechanism, a second physical force larger than the first physical force to the content of the culture vessel via the second liquid to detach the second cell present in the culture vessel. The collection step is a step of collecting from the culture vessel, by a collection mechanism, the second liquid containing the detached second cell.

According to a second aspect of the present disclosure, a cell extraction system includes a first detachment mechanism, a second detachment mechanism, and a collection mechanism. The first detachment mechanism is configured to detach at least a part of a first cell from a culture vessel, wherein contents of the culture vessel comprise the first cell and a second cell which has a higher adhesion strength than the first cell which coexist in the culture vessel, by applying a first physical force to the contents of the culture vessel via a first liquid after the first and second cells are immersed in the first liquid containing a detachment reagent. The second detachment mechanism is configured to detach the second cell present in the culture vessel by applying a second physical force larger than the first physical force to the content of the culture vessel via a second liquid injected into the culture vessel after the first liquid containing the first cell detached is discharged from the culture vessel. The collection mechanism is configured to collect from the culture vessel the second liquid containing the second cell detached.

Features of the present disclosure will become apparent from the following description of embodiments with reference to the attached drawings. The following description of embodiments is described by way of example.

### BRIEF DESCRIPTION OF THE DRAWINGS

FIG. 1 is a block diagram illustrating a configuration of a cell detachment and selection system including a cell detachment device according to an embodiment.

FIG. 2 is a schematic diagram illustrating a configuration of the cell detachment device according to the embodiment.

FIG. 3 is a flowchart illustrating a procedure for selectively collecting a second cell by detaching a cell from a culture vessel by the cell detachment and selection system.

FIG. 4A is a schematic diagram for describing step S1.

FIG. 4B is a schematic diagram for describing steps S2 and S3.

FIG. 4C is a schematic diagram for describing steps S4 and S5.

FIG. 5A is a schematic diagram for describing step S6.

FIG. 5B is a schematic diagram for describing step S7.

FIG. 5C is a schematic diagram for describing steps S8 and S9.

FIG. 6A is a schematic diagram for describing step S10.

FIG. 6B is a schematic diagram for describing step S11.

### DESCRIPTION OF THE EMBODIMENTS

A cell extraction method, a cell extraction system, and the like according to an embodiment of the present disclosure will be described with reference to the drawings. The embodiments described below are merely examples, and for example, detailed configurations can be appropriately changed and implemented by those skilled in the art without departing from the gist of the present disclosure.

In the drawings referred to in the following description of the embodiments and examples, elements denoted by the same reference signs have the same functions unless otherwise specified. In the drawings, in a case where a plurality of the same elements are arranged, reference signs and a description thereof may be omitted.

In addition, the drawings may be schematic for convenience of illustration and description, and thus, the shape, size, arrangement, and the like of elements in the drawings may not strictly match those of actual ones. In addition, “XX or more and YY or less” or “XX to YY” representing a numerical range means a numerical range including end points XX (lower limit) and YY (upper limit) unless otherwise specified. When numerical ranges are described in stages, the upper limit and the lower limit of each numerical range can be arbitrarily combined.

### EMBODIMENT

### Configuration of Cell Extraction System

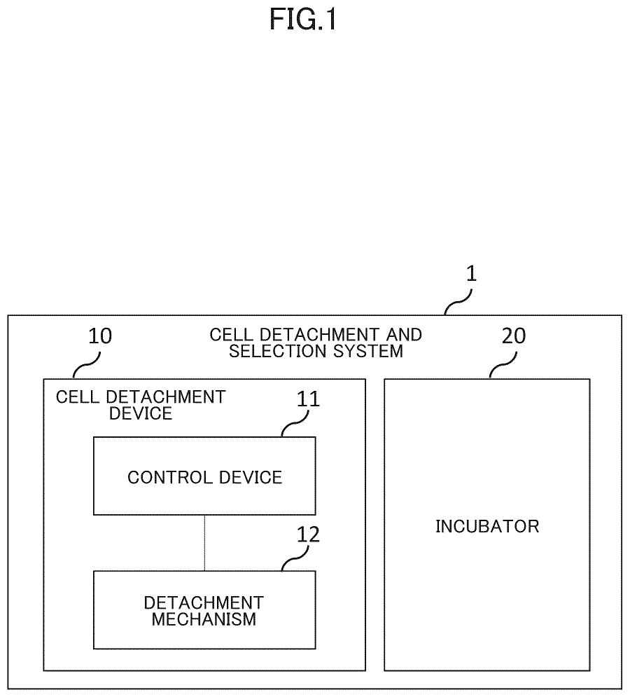

FIG. 1 is a block diagram illustrating a configuration of a cell detachment and selection system 1 serving as a cell extraction system according to an embodiment. The cell detachment and selection system 1 illustrated in FIG. 1 is a cell extraction system capable of performing a cell extraction method according to the embodiment, and selectively detaches a target cell from among two or more types of cells and extracts the target cell from a culture vessel. As illustrated in FIG. 1, the cell detachment and selection system 1 includes a cell detachment device 10 and an incubator 20.

A cell handled in the present embodiment is not particularly limited as long as the cell can be applied to adherent culture using the culture vessel. For example, the cell can be a mammalian cell such as a primate cell or a rodent cell, and may be a cell of a human, a monkey, a mouse, a rat, a guinea pig, a hamster, a rabbit, a cat, a dog, a sheep, a pig, a cow, or a goat, or a cell derived therefrom.

The cell handled by the cell extraction system according to the present embodiment may be a pluripotent stem cell, and examples of the pluripotent stem cell include an embryonic stem cell (ES cell), an induced pluripotent stem cell (iPS cell or artificial pluripotent stem cell), a multilineage-differentiating stress enduring cell (Muse cell), an embryonal carcinoma cell (EC cell), and an embryonic germ cell (EG cell). In the present embodiment, a cell to be extracted can be a pluripotent stem cell of a mammal such as a primate or a rodent. The cell to be extracted can be an induced pluripotent stem (iPS) cell of a mammal such as a primate or a rodent, and can be a human iPS cell. In the present embodiment, either a cell that proliferates by forming a single layer on an adhesion surface or a cell that proliferates by forming a plurality of layers may be used.

The cell detachment device 10 is a mechanical device that detaches and collects the cell cultured in the culture vessel from the culture vessel, and includes a control device 11 and a detachment mechanism 12. The control device 11 is a computer that comprehensively controls an operation of the detachment mechanism 12, and the detachment mechanism 12 operates according to a command from the control device 11. The control device 11 may be a part of a control unit that controls the entire cell detachment and selection system 1 including the incubator 20.

The detachment mechanism 12 is a mechanical mechanism having a function of detaching, from the culture vessel, the cell cultured in the culture vessel. As described in detail below, the detachment mechanism 12 can apply a physical force to a content of the culture vessel via a liquid to facilitate detachment of the cell. When detaching a first cell from the culture vessel, the detachment mechanism 12 can apply a first physical force to the content of the culture vessel via a flowing first liquid. In addition, when detaching a second cell from the culture vessel, the detachment mechanism 12 can apply a second physical force larger than the first physical force to the content of the culture vessel via a flowing second liquid. The detachment mechanism 12 may apply the first physical force and the second physical force by another type of mechanism, or may apply the first physical force and the second physical force by changing an operation condition using the same type of mechanism.

The incubator 20 includes a chamber capable of accommodating the culture vessel, and can culture the cell accommodated in the culture vessel by controlling a temperature and/or humidity in the chamber. The incubator 20 and the cell detachment device 10 may be implemented separately or integrally. In the latter case, as an example, it can be said that the cell can be cultured by the cell detachment device 10 if all or some of components of the detachment mechanism 12 are provided in the chamber of the incubator 20.

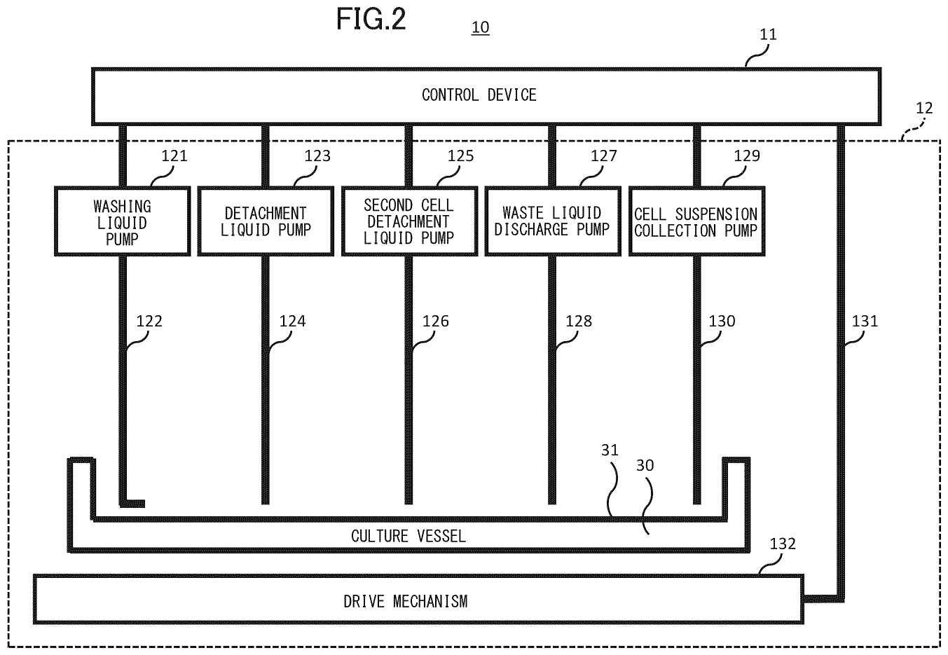

FIG. 2 is a schematic diagram illustrating a configuration of the cell detachment device 10 according to the embodiment. A culture vessel 30 in which the cell is cultured is disposed in the detachment mechanism 12. A shape of the culture vessel 30 is not particularly limited, but a dish, a petri dish, a flask, a well plate, or the like having an opening through which the detachment mechanism 12 can access the inside of the culture vessel 30 is suitably used. An inner bottom surface of the culture vessel 30 is referred to as a culture surface 31. The cultured cell is in contact with the culture surface 31. Before cell detachment processing performed by the detachment mechanism 12, a plurality of cell masses (colonies) are formed in the culture vessel 30 and adhere to the culture surface 31.

The culture vessel 30 is an open-type culture vessel or a closed-type culture vessel having the culture surface 31 suitable for adherent culture of the cell. A structure and a material of the culture surface 31 are not particularly limited as long as the culture surface 31 is suitable for cell culture, a coating for cell adhesion can be applied in order to improve cell adhesion efficiency. Examples of the coating for cell adhesion include extracellular matrix (ECM) coatings such as gelatin, collagen, laminin, fibronectin, entactin, hyaluronic acid, adhesive oligopeptides, polylactic acid, polyglycolic acid, poly-L-lysine, or poly-L-ornithine, and polymer coatings. In particular, as the culture vessel 30 for pluripotent stem cells, for example, a 35 mm dish 430165 manufactured by CORNING (registered trademark) can be used as the culture vessel, and the culture surface 31 can be formed by coating the surface thereof with, for example, iMatrix-551 (registered trademark) manufactured by MATRIXOME.

The cell detachment device 10 sequentially detaches two or more types of cells adherently cultured in the culture vessel 30 from the culture vessel 30. In the present embodiment, for example, a partially reprogrammed cell whose reprogramming has been started using a viral vector and an iPS cell can be handled as the two or more types of cells, and can be sequentially detached. When comparing the partially reprogrammed cell with the iPS cell, an adhesion strength of the iPS cell is higher than that of the partially reprogrammed cell, and in the present embodiment, the iPS cell is detached at a high selection ratio and extracted using such a difference in adhesion strength as described in detail below.

In the following description of the embodiment, it is assumed that the cell cultured in the culture vessel 30 is a cell obtained in reprogramming a mononuclear cell of a single donor, the first cell is a GFP-positive cell whose reprogramming has not been completed, and the second cell is the iPS cell whose reprogramming has been completed.

As illustrated in FIG. 2, the cell detachment device 10 includes the control device 11 and the detachment mechanism 12. The detachment mechanism 12 includes various pumps such as a washing liquid pump 121, a detachment liquid pump 123, a second cell detachment liquid pump 125, a waste liquid discharge pump 127, and a cell suspension collection pump 129, and a drive mechanism 132. The control device 11 is communicably connected to the detachment mechanism 12 in a wired or wireless manner, and controls operations of various pumps and the drive mechanism 132 included in the detachment mechanism 12.

In response to a control signal from the control device 11, the washing liquid pump 121 delivers a washing liquid into the culture vessel 30 via a washing liquid delivery tube 122. The washing liquid delivery tube 122 is a cylindrical structure (such as a tube) through which the washing liquid flows. The washing liquid is used to wash away impurities adhering to the culture surface 31 of the culture vessel 30 and the cell present in the culture vessel 30. Examples of the washing liquid include physiological saline, a liquid culture medium, and a cryopreservation liquid stored in a washing liquid tank (not illustrated).

The washing liquid pump 121 sucks the washing liquid from the washing liquid tank and delivers the washing liquid into the culture vessel 30 via the washing liquid delivery tube 122. As a result, the culture surface 31 and/or the cell adhering to the culture surface 31 can be washed. The impurities removed by washing are, for example, dead cells floating in the culture vessel 30 or calcium ions and magnesium ions present in a culture medium of the culture vessel 30. If the washing is insufficient, calcium ions and magnesium ions remaining in the culture medium may react with a chelating agent when a detachment reagent is injected, resulting in a possibility that the chelating agent cannot sufficiently cleave intercellular adhesion. In the present embodiment, sufficient washing is performed by a washing unit including the washing liquid pump 121 and the washing liquid delivery tube 122.

In response to a control signal from the control device 11, the detachment liquid pump 123 ejects a liquid containing the detachment reagent (first liquid) into the culture vessel 30 via a detachment reagent delivery tube 124 (first injection unit). The detachment reagent delivery tube 124 is a cylindrical structure (such as a tube) through which a liquid containing the detachment reagent flows. The detachment reagent has an action of weakening adhesion between the cell and the culture surface 31 and adhesion between the cells. As the liquid (first liquid) containing the detachment reagent, for example, a solution containing a proteolytic enzyme and/or the chelating agent stored in a detachment liquid tank (not illustrated) can be used. As described below, the detachment reagent is used, for example, to detach the GFP-positive cell, which is the first cell, from the culture vessel 30.

The detachment liquid pump 123 sucks the detachment liquid from a detachment reagent tank and delivers the detachment liquid to the culture vessel 30 via the detachment reagent delivery tube 124. As a result, the cell adhering to the culture surface 31 is immersed in the liquid (first liquid) containing the detachment reagent.

In response to a control signal from the control device 11, the second cell detachment liquid pump 125 ejects a second cell detachment liquid (second liquid) into the culture vessel 30 via a second cell detachment liquid ejection tube 126 (second injection unit). The second cell detachment liquid ejection tube 126 is a structure that supplies and ejects the second cell detachment liquid, and for example, a tube, a pipette, or a spray nozzle is used. The second cell detachment liquid is used for detaching the second cell that is in contact with the culture surface 31 from the culture surface 31. As the second cell detachment liquid, a liquid that does not contain the proteolytic enzyme or the chelating agent is used, and physiological saline or a liquid culture medium can be used.

The second cell detachment liquid pump 125 sucks the second cell detachment liquid from a second cell detachment liquid tank (not illustrated), and ejects the second cell detachment liquid to the culture vessel 30 via the second cell detachment liquid ejection tube 126. The second cell detachment liquid pump 125 and the second cell detachment liquid ejection tube 126 form an ejection mechanism that operates according to a control signal from the control device 11.

The second cell that is in contact with the culture surface 31 is detached from the culture surface 31 by an action of a physical force applied by the second cell detachment liquid. The detachment mechanism 12 can apply the first physical force via the first liquid when detaching the first cell from the culture vessel, and can apply the second physical force larger than the first physical force via the second liquid when detaching the second cell from the culture vessel. In this example, by appropriately setting an operation condition (for example, an ejection pressure or a flow velocity) of the second cell detachment liquid pump 125 and a structure of an outlet of the second cell detachment liquid ejection tube 126, the second physical force larger than the first physical force can be applied to the cell in the culture vessel via the second liquid. In this example, a second detachment mechanism is implemented by the ejection mechanism including the second cell detachment liquid pump 125 and the second cell detachment liquid ejection tube 126.

In response to a control signal from the control device 11, the waste liquid discharge pump 127 sucks a liquid (waste liquid) to be discharged from the culture vessel 30 via a waste liquid suction tube 128. The waste liquid may be, for example, the liquid (detachment liquid) containing the detachment reagent remaining in the culture vessel 30, the washing liquid, or the liquid culture medium. The waste liquid suction tube 128 is a cylindrical structure that sucks and circulates the waste liquid, and for example, a tube is used. The waste liquid sucked from the culture vessel 30 is discharged to a waste liquid tank (not illustrated).

In response to a control signal from the control device 11, the cell suspension collection pump 129 sucks a cell suspension from the culture vessel 30 via a cell suspension suction tube 130 and collects the cell suspension in a collection tank (not illustrated). The cell suspension is the second cell detachment liquid containing the second cell, which is removed from the culture surface 31. The cell suspension collection pump 129, the cell suspension suction tube 130, and the collection tank (not illustrated) form a collection mechanism.

The drive mechanism 132 is a mechanical mechanism capable of changing a posture of the culture vessel 30 in response to a control signal input from the control device 11 via a signal line 131. Although a specific form of the drive mechanism 132 is not particularly limited, for example, the drive mechanism 132 can be implemented by a drive source such as a motor or an air cylinder, and a movable portion in which a link and a cam are combined to support the culture vessel 30 such that the posture of the culture vessel 30 can be changed. By appropriately setting a driving condition (for example, a tilting angle of the culture vessel in a rocking motion, a cycle of the rocking motion, or the number of rocking cycles) of the drive mechanism 132, the first physical force can be applied to the cell in the culture vessel via the first liquid. In this example, a first detachment mechanism is implemented by the drive mechanism 132.

By executing a control program recorded in advance in a recording medium, the control device 11 controls the operations of each pump and the drive mechanism 132 described above to automatically perform processing of detaching and collecting a desired cell from the culture vessel. For example, the control device 11 can perform control of a sequence of ejecting or sucking various types of liquids, control of a flow rate, control of a timing, and the like.

With the cell detachment and selection system 1 according to the present embodiment, it is possible to selectively collect cells that have a small difference in adhesion strength and are present in the culture vessel at a high yield. For example, the iPS cell can be collected at a high selection ratio from the culture vessel in which the partially reprogrammed cell in which a reprogramming factor has been introduced into the mononuclear cell and the iPS cell in which the reprogramming factor has been introduced into the mononuclear cell and of which reprogramming has been completed coexist. For example, it is possible to sequentially detach the partially reprogrammed cell whose reprogramming has been started using the viral vector and the iPS cell, and to collect the iPS cell at a high selection ratio.

### Cell Detachment Method and Cell Extraction Method

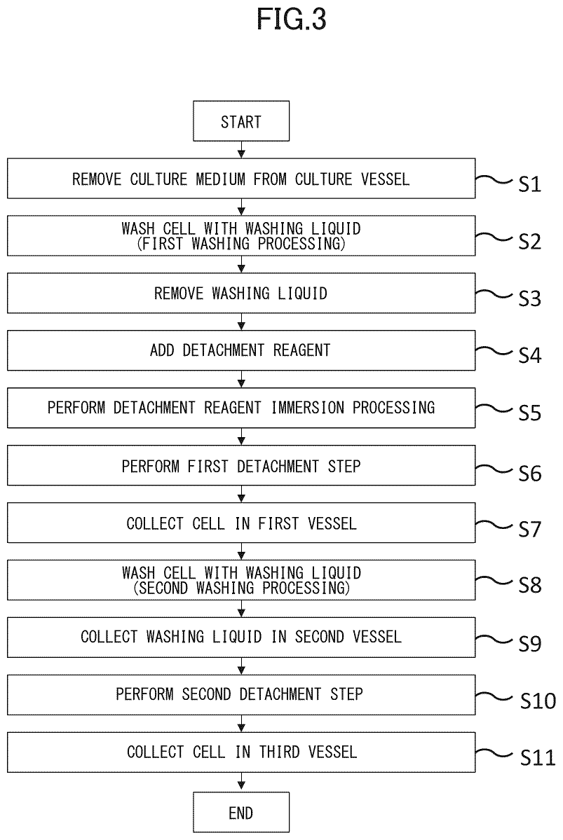

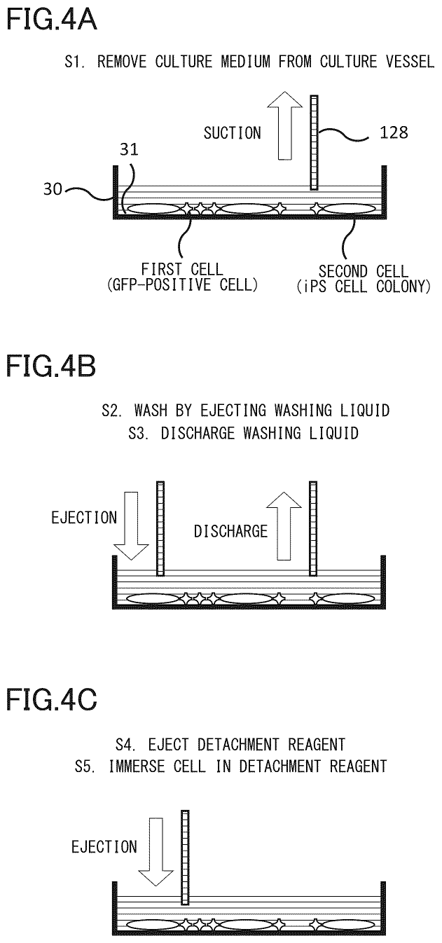

A processing procedure for selective detachment and collection of the cell by the cell detachment and selection system 1 will be described with reference to the drawings. FIG. 3 is a flowchart illustrating a procedure of the cell extraction method of selectively extracting the second cell by detaching the cell from the culture vessel by the cell detachment and selection system 1. FIGS. 4A to 6B are schematic diagrams for describing steps of the processing procedure. In each drawing, only configurations necessary for description of the steps are illustrated, and illustration of configurations not necessary for description is appropriately omitted.

In the following example, it is assumed that the incubator 20 and the cell detachment device 10 are integrally formed. Specifically, the washing liquid delivery tube 122, the detachment reagent delivery tube 124, the second cell detachment liquid ejection tube 126, the waste liquid suction tube 128, the cell suspension suction tube 130, and the drive mechanism 132 are disposed in the chamber of the incubator 20 so as to be accessible to the culture vessel.

Before starting cell extraction processing, the culture vessel 30 accommodates the GFP-positive cell which is the cultured first cell, a colony of the iPS cells which are the cultured second cells, and the culture medium used for the culture. The cells in the culture vessel 30 may be cultured in the incubator 20 or may be cultured in another incubator.

When the cell extraction processing of selectively detaching and collecting the second cell from among the first cell and the second cell coexisting in the culture vessel 30 is started, first, in step S1, the liquid culture medium is removed from the culture vessel 30 as illustrated in FIG. 4A. Specifically, the control device 11 drives the waste liquid discharge pump 127. The waste liquid discharge pump 127 sucks the liquid culture medium from the inside of the culture vessel 30 via the waste liquid suction tube 128 and discharges the liquid culture medium to the outside of the culture vessel 30. The discharged waste liquid is accommodated in the waste liquid tank (not illustrated). The first cell, the second cell, the culture medium that has not been sucked, and the like remain in the culture vessel 30.

Next, in step S2, first washing processing (first washing step) is performed. As illustrated in FIG. 4B, the control device 11 drives the washing liquid pump 121 to deliver the washing liquid into the culture vessel 30 via the washing liquid delivery tube 122. In addition to the adherently cultured cell, the culture medium remaining without being sucked in step S1 and the impurities such as nutrient components contained in the culture medium remain in the culture vessel 30, but the impurities are washed away from the cell by the washing liquid ejected from the washing liquid delivery tube 122.

Subsequently, in step S3, as illustrated in FIG. 4B, the control device 11 drives the waste liquid discharge pump 127 to suck the washing liquid containing the impurities via the waste liquid suction tube 128, discharge the washing liquid from the culture vessel 30, and accommodate the discharged washing liquid in the waste liquid tank (not illustrated). By steps S2 and S3, the first washing processing is performed, and the cell in the culture vessel 30 and the culture surface 31 of the culture vessel 30 are washed.

Subsequently, in step S4 serving as a detachment reagent injection step, the control device 11 drives the detachment liquid pump 123 to eject the detachment reagent (first liquid) into the culture vessel 30 via the detachment reagent delivery tube 124 as illustrated in FIG. 4C. An amount of detachment reagent sufficient to immerse the first and second cells is ejected into the culture vessel 30.

Subsequently, in step S5, as illustrated in FIG. 4C, in order to cause the detachment reagent to act, immersion processing (immersion step) of immersing the first cell and the second cell in the detachment reagent for a predetermined time is performed. By the action of the detachment reagent, both the adhesion strength of the GFP-positive cell (first cell) and the adhesion strength of the iPS cell (second cell) can be reduced. The immersion processing is performed to make the adhesion strength of the first cell become lower than the adhesion strength of the second cell, so that the first cell is likely to be selectively detached by first detachment processing described below.

It is also possible to perform incubation in parallel with the immersion processing. In the case of performing incubation, the control device 11 notifies the incubator 20 of an incubation start command. Upon receiving the start command, the incubator 20 maintains an environment in which the culture vessel 30 to which the detachment liquid is added is placed at a predetermined temperature and humidity for a predetermined period. It is sufficient if the predetermined temperature and humidity are set to conditions suitable for culturing the iPS cell. For example, it is sufficient if the temperature is set to about 37° C. and the humidity is set to about 95%. The detachment liquid acts during incubation, and the adhesion strength between the GFP-positive cell (first cell) and the culture surface 31, the adhesion strength between the iPS cells (second cells), and the adhesion strength between the iPS cell (second cell) and the culture surface 31 are reduced. It is sufficient if the period (incubation period) during which the predetermined temperature is maintained is set to have a length determined by experiments, for example, as a time required to reduce the adhesion strength to a predetermined degree. After the elapse of the incubation period, the control device 11 ends the control of the temperature and the humidity by the incubator 20.

In a case where the incubation is not performed in parallel with the immersion processing, the control device 11 immerses the first cell and the second cell in the detachment reagent for a predetermined immersion period. It is sufficient if the immersion period is set to have a length determined by experiments, for example, as a time required to reduce the adhesion strength to a predetermined degree.

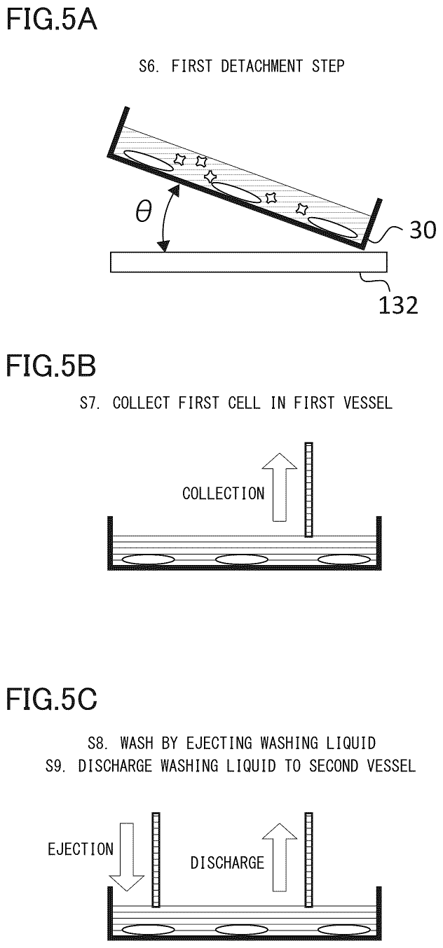

After the immersion processing, in step S6 serving as a first detachment step, the first detachment processing of selectively detaching the GFP-positive cell (first cell) is performed. In the first detachment processing, as illustrated in FIG. 5A, the posture of the culture vessel 30 is changed by the drive mechanism 132, and the first physical force is applied to the content of the culture vessel 30 via a detachment reagent solution (first liquid) flowing due to the posture change to detach at least a part of the first cell. Although FIG. 5A is a schematic diagram and thus a liquid surface appears not to be inclined, the liquid surface may actually be inclined. For example, by repeatedly tilting the culture vessel 30 and causing the detachment liquid to flow, the first cell which is the GFP-positive cell is selectively detached from the culture vessel or the second cell.

Step S6 is desirably performed immediately after step S5 is completed. This is because it is possible to prevent the second cell from being unnecessarily continuously immersed in the detachment liquid, and to reduce a possibility that the second cell is damaged by the detachment liquid.

The first detachment processing may be performed in the closed culture vessel 30. With the closed culture vessel 30, even if the liquid surface shakes, the liquid does not overflow to the outside of the culture vessel 30 and does not contaminate the device. Further, the liquid overflowing from the culture vessel 30 does not come into contact with an external environment and then flow back to the culture vessel 30, thereby preventing contamination from being introduced into the culture vessel 30. However, in a case where the flow of the liquid in the culture vessel 30 is controlled with high accuracy, for example, the first detachment processing may be performed in an open culture vessel without a lid.

Conditions for performing the first detachment processing are set such that the first cell is sufficiently detached and the detachment of the second cell is suppressed. For example, it is sufficient if a tilting angle θ of a bottom surface of the culture vessel with respect to a horizontal plane is set to 5°, the number of rocking cycles is set to 20, a time required for one tilting operation is set to 0.3 seconds, and an interval between the tilting operations is set to 0.5 seconds. For example, the tilting operation is controlled such that an average flow speed of the detachment liquid moving inside the culture vessel 30 by the operation of tilting the culture vessel 30 becomes 0.24 (m/sec).

The first detachment processing is desirably performed under a detachment condition that 70% or more of the first cells present in the culture vessel 30 at the start of the detachment reagent injection step (step S4) are detached.

For example, a state of the culture surface 31 during or after the first detachment processing is observed using a camera or the like, and the tilting angle, a tilting speed, the number of tilting operations, the interval between the tilting operations, and the like may be adjusted such that the first cell is sufficiently detached but the detachment of the second cell is suppressed. Such conditions may be set in advance by experiments and stored as operation parameters of the control program of the control device 11.

In subsequent step S7, the control device 11 drives the waste liquid discharge pump 127 to collect the detachment reagent from the culture vessel 30 via the waste liquid suction tube 128 as illustrated in FIG. 5B, and discharges the detachment reagent to a first vessel (not illustrated). The collected detachment reagent includes the cells (mainly the first cells) detached in the first detachment processing, and when the collected detachment reagent is handled as the waste liquid, the first vessel may be the waste liquid tank. In addition to the second cell to be extracted, the impurities such as the detachment reagent and the first cell that have not been sucked may remain in the culture vessel 30.

In subsequent step S8, second washing processing (second washing step) is performed. As illustrated in FIG. 5C, the control device 11 drives the washing liquid pump 121 to eject the washing liquid into the culture vessel 30 via the washing liquid delivery tube 122. The washing liquid pump 121 and the washing liquid delivery tube 122 form the washing unit.

As the content of the culture vessel 30, in addition to the undetached second cell, there are impurities such as the detachment reagent remaining without being sucked in step S7, the first cell present in the detachment reagent, and the first cell adhering to the second cell. The undetached second cell is washed by the ejected washing liquid to wash off the impurities from the second cell.

In order to effectively perform the second washing processing, the control device 11 can rock the culture vessel 30 by operating the drive mechanism 132 simultaneously with the ejection of the washing liquid or after the ejection of the washing liquid. As a result, the impurities such as the first cell adhering to the culture vessel 30 and the first cell adhering to the second cell can be efficiently removed. The operation of the drive mechanism 132 is performed under a condition that the first cell (GFP-positive cell) adhering to the second cell (iPS cell) and the detachment reagent remaining in the culture vessel 30 are sufficiently washed away. For example, it is sufficient if the tilting angle θ of the bottom surface of the culture vessel with respect to the horizontal plane is set to 5°, the number of rocking cycles is set to 20, the time required for one tilting operation is set to 0.3 seconds, and the interval between the tilting operations is set to 0.5 seconds. For example, the tilting operation is controlled such that an average flow speed of the washing liquid moving inside the culture vessel 30 by the operation of tilting the culture vessel 30 becomes 0.24 (m/sec).

The second washing processing is desirably performed under a washing condition that 70% or more of the first cells present in the culture vessel 30 at the start of the detachment reagent injection step (step S4) are removed from the inside of the culture vessel 30. In addition, the second washing processing is desirably performed under a washing condition that 70% or more of the second cells present in the culture vessel 30 at the start of the detachment reagent injection step (step S4) remain inside the culture vessel 30 at the end of the second washing processing.

For example, the state of the culture surface 31 during or after the second washing processing is observed using a camera or the like, and the tilting angle, the tilting speed, the number of tilting operations, the interval between the tilting operations, and the like may be adjusted such that the impurities are sufficiently washed away, but the detachment of the second cell is suppressed. Such washing conditions may be set in advance by experiments and stored as operation parameters of the control program of the control device 11.

Step S8 is desirably performed immediately after step S7 is completed. It is possible to prevent the second cells from being unnecessarily kept in contact with the detachment liquid remaining in the culture vessel 30, and to reduce a possibility that the second cell is damaged by the detachment liquid.

Then, in step S9, as illustrated in FIG. 5C, the control device 11 drives the waste liquid discharge pump 127 to suck the washing liquid containing the impurities via the waste liquid suction tube 128, discharge the washing liquid from the culture vessel 30, and accommodate the discharged washing liquid in a second vessel (not illustrated). In a case where the collected washing liquid is handled as the waste liquid, the second vessel may be the waste liquid tank.

By performing steps S8 and S9, the undetached second cell present in the culture vessel 30 and the culture surface 31 of the culture vessel 30 are washed. In order to effectively remove the detachment reagent remaining in the culture vessel 30 and the first cell adhering to the second cell, steps S8 and S9 of the flowchart of FIG. 3 may be repeatedly performed a plurality of times (for example, twice).

In subsequent step S10, second detachment processing (second detachment step) is performed. In the second detachment processing, a physical force is applied to the content of the culture vessel 30 via the second cell detachment liquid (second liquid) to detach the second cell. In the first detachment processing, the first physical force was applied to the content of the culture vessel 30 via the detachment reagent (first liquid) flowing due to the posture change of the culture vessel 30. In the second detachment processing, the second physical force larger than the first physical force in the first detachment processing is applied to the second cell via the second cell detachment liquid (second liquid) to detach the second cell. As the second cell detachment liquid, a liquid that does not adversely affect the characteristics of the second cell is used, and for example, physiological saline or a liquid culture medium is used. That is, a liquid containing no detachment reagent, such as the proteolytic enzyme or the chelating agent, is used.

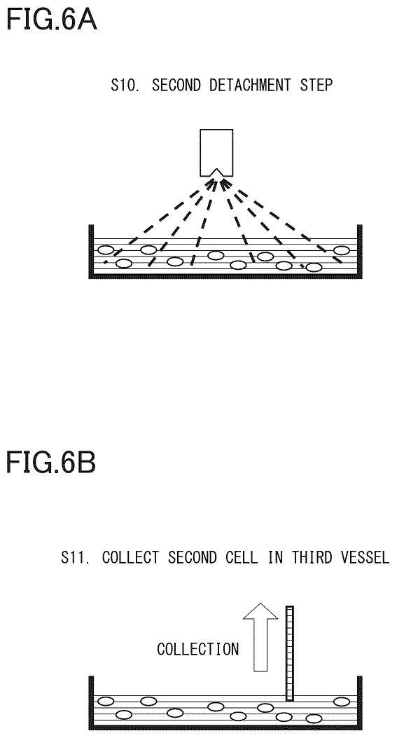

Specifically, as illustrated in FIG. 6A, the control device 11 drives the second cell detachment liquid pump 125 to eject the second cell detachment liquid (second liquid) toward the culture surface 31 in a mist form via the second cell detachment liquid ejection tube 126. The second cell detachment liquid is ejected under such ejection conditions that the second cell can be detached but is not physically damaged.

A tip of the second cell detachment liquid ejection tube 126 can be a spray nozzle, and the second cell detachment liquid is ejected as a plurality of fine droplets having a predetermined initial speed. An average particle diameter of the fine droplets is, for example, 50 μm or more and 600 μm or less. That is, the “mist form” does not refer to a shape of a spatial range (such as a conical range or a linear range) in which the droplets are ejected, but indicates that the droplets are minute in size.

The droplets of the second cell detachment liquid may be continuously ejected from the second cell detachment liquid ejection tube 126, or may be ejected in a pulsed or intermittent manner. For example, the second cell may be detached from the culture vessel 30 by ejecting a droplet group of the second cell detachment liquid once from the second cell detachment liquid ejection tube 126. At this time, a single ejection amount of the second cell detachment liquid, that is, the total amount of the droplet group ejected in one ejection may be, for example, 3.0 mL or less. A single ejection time of the second cell detachment liquid may be, for example, 0.2 seconds or less. An ejection angle from the second cell detachment liquid ejection tube 126 can be set such that the ejected droplet group of the second cell detachment liquid reaches (contacts) the entire surface (for example, an area of 95% or more) of the second cell colony in plan view. Typically, the second cell detachment liquid ejection tube 126 ejects the second cell detachment liquid in a conical range. However, the second cell detachment liquid ejection tube 126 may also eject the second cell detachment liquid linearly in a vertical direction, for example.

By a mechanical action of the ejected droplets of the second cell detachment liquid, a shearing force stronger than that in the first detachment processing (step S6) is directly applied to the second cell (iPS cell), so that the second cell (iPS cell) is detached from the culture surface 31. As a result, the detached iPS cell is suspended in the second cell detachment liquid ejected into the culture vessel 30, and the second cell detachment liquid becomes the cell suspension.

The second detachment processing is desirably performed under a condition that 90% or more of the second cells present in the culture vessel 30 at the end of the second washing processing (step S10) are detached from the culture vessel 30 and contained in the cell suspension.

Droplet ejection conditions (for example, the size of the droplet, an ejection frequency, and the initial speed of the droplet) may be adjusted such that the second cell is appropriately detached by observing the state of the culture surface 31 during or after the second detachment processing using, for example, a camera. Such ejection conditions may be set in advance by experiments and stored as operation parameters of the control program of the control device 11 (for example, drive conditions of the second cell detachment liquid pump 125).

Once the second detachment step (step S10) is completed, the processing proceeds to step S11 serving as a collection step, and the cell suspension in which the second cell (iPS cell) to be extracted is suspended is collected from the culture vessel 30. Specifically, as illustrated in FIG. 6B, the control device 11 drives the cell suspension collection pump 129 to suck the cell suspension from the culture vessel 30 via the cell suspension suction tube 130, and injects the cell suspension into a third vessel (not illustrated) detachably mounted on the cell detachment and selection system 1. The cell suspension may be injected into one third vessel, or the cell suspension may be separately injected into a plurality of third vessels. The third vessel storing the cell suspension of the iPS cell that is a target is removed from the cell detachment and selection system 1, and is conveyed to a device or instrument of the next step (for example, subculture) according to the purpose of use of the iPS cell.

By performing the above steps, the cell detachment processing performed by the cell detachment and selection system 1 is completed. With the cell detaching method according to the present embodiment, it is possible to distinguish cells that have a small difference in adhesion strength and are present in the culture vessel and selectively collect the cells at a high yield. For example, the iPS cell can be collected at a high selection ratio from the culture vessel in which the partially reprogrammed cell in which a reprogramming factor has been introduced into the mononuclear cell and the iPS cell in which the reprogramming factor has been introduced into the mononuclear cell and of which reprogramming has been completed coexist. For example, it is possible to sequentially detach the partially reprogrammed cell whose reprogramming has been started using the viral vector and the iPS cell, and to collect the iPS cell at a high selection ratio.

### EXAMPLE

In the following Example, in a subculture system in which an iPS cell is produced from a mononuclear cell and subcultured, the cell extraction device and the cell extraction method according to the above-described embodiment were used. Cells cultured in a culture vessel are cells obtained by reprogramming a mononuclear cell of a single donor, in which the first cell is the GFP-positive cell and the second cell is the iPS cell.

In Example, a cell suspension containing the iPS cell which is the second cell was collected by the method described with reference to FIG. 3 and FIGS. 4A to 6B. In Comparative Example, the first detachment step in step S6 described in FIG. 3 was not performed, and only the second detachment step in step S10 was performed to collect the cell suspension from the culture vessel.

For Example and Comparative Example, subculture from P0 to P1 was performed based on the cell suspension collected from the culture vessel. First, for P0, the cells were detached by the methods of Example and Comparative Example, and a ratio of the GFP-positive cells to all cells contained in the cell suspension collected in step S11 was measured. By using the collected cell suspension, the cells were re-seeded in a new culture vessel and cultured in a culture unit for seven days. Thereafter, detachment and collection were performed on the new culture vessel by the methods of Example and Comparative Example, and the ratio of GFP-positive cells to all cells was measured. A reduction rate of the GFP-positive cells was calculated for Example and Comparative Example by using a GFP-positive cell ratio at the time of P0 cell collection and a GFP-positive cell ratio at the time of P1 cell collection. The reduction rate is defined by the following Formula 1.

Reduction Rate=(GFP-Positive Cell Content at P1)/(GFP-Positive Cell Content at P0) Formula 1

Table 1 shows the reduction rate of the GFP-positive cells after subculture from P0 to P1 in Example and Comparative Example.

TABLE 1

| | Comparative | |
|---|---|---|
| | Example | Example |
| Reduction Rate of GFP-Positive Cells | −37.5% | −90.7% |
| after Subculture (P0 → P1) | | |

As shown in Table 1, in subculture, a reduction rate of the GFP-positive cells in Example is extremely higher than that in Comparative Example. In Example, in the first detachment processing, the GFP-positive cell having a relatively low adhesion strength is selectively discharged from the culture vessel, and thereafter, the iPS cell having a relatively high adhesion strength and remaining in the culture vessel is collected by the second detachment processing. Therefore, the reduction rate of the GFP-positive cells is higher than that in Comparative Example.

In Example, a cell that does not exhibit pluripotency during reprogramming can be removed at a high selection ratio before differentiation induction. Therefore, the purity of the iPS cells, which are the target of subculture, can be increased, and it can be seen that the quality of the extracted iPS cells in Example is extremely superior to that in Comparative Example.

### Modified Embodiment

The present disclosure is not limited to the embodiments and examples described above, and many modifications can be made within the technical idea of the present disclosure. For example, all or some of the different embodiments and examples described above may be combined and implemented.

For example, the first cell is not limited to the GFP-positive cell, and may be, for example, an SSEA-1-positive cell.

The washing liquid collected from the culture vessel in step S3, the detachment reagent collected from the culture vessel in step S7, and the washing liquid collected from the culture vessel in step S9 may be collected in another vessel according to a processing method after collection, or may be collected in a common vessel (for example, the waste liquid tank).

In the first detachment step (step S6), it is sufficient if the weak shearing force (first physical force) that detaches the first cell but does not detach the second cell in the culture vessel 30 can be applied to the vicinity of the culture surface 31 via the first liquid. Therefore, the first detachment step is not limited to the method/mechanism of tilting the culture vessel 30. For example, a mechanism of moving the culture vessel 30 on the horizontal plane (for example, linear reciprocating motion and/or circular motion in plan view) without tilting the culture surface 31 of the culture vessel 30 with respect to a horizontal direction may be used. Alternatively, a mechanism of vertically moving the culture vessel 30 may be used. Alternatively, tilting of the culture vessel 30, movement in the horizontal direction, movement in the vertical direction, application of vibration (for example, ultrasonic vibration) to the culture vessel, and the like may be combined to apply the weak shearing force (first physical force).

In addition, the first liquid may flow in the culture vessel 30 by a method such as providing a flow mechanism or a stirring mechanism in the culture vessel 30 or using a pump installed outside the culture vessel 30 to apply the weak shearing force (first physical force) to the vicinity of the culture surface 31. For example, the flow of the detachment reagent may be generated in the culture vessel 30 by operating the waste liquid discharge pump 127 to suck the detachment reagent via the waste liquid suction tube 128 while ejecting the detachment reagent from the detachment liquid pump 123 via the detachment reagent delivery tube 124.

The second detachment mechanism may apply the second physical force by using a mechanism different from the first detachment mechanism, or may apply the second physical force by changing an operation condition using the same mechanism as the first detachment mechanism. For example, after the second liquid is injected into the culture vessel, the same mechanism as the first detachment mechanism may rock the culture vessel in the second detachment step with a greater intensity than the first detachment step.

The control device 11 is a computer that comprehensively controls the detachment mechanism 12, and includes a processor. The processor may be implemented by, for example, a central processing unit (CPU), a graphics processing unit (GPU), an application specific integrated circuit (ASIC), or a programmable logic device (for example, a simple programmable logic device (SPLD), a complex programmable logic device (CPLD), or a field programmable gate array (FPGA)). The processor implements the function by reading and executing a program stored in a storage circuit. Instead of storing the program in the storage circuit, the program may be directly incorporated in a circuit of the processor. In this case, the processor implements the function by reading and executing the program incorporated in the circuit. On the other hand, in a case where the processor is, for example, an ASIC, the function is directly incorporated as a logic circuit in the circuit of the processor instead of storing the program in the storage circuit. The processor can perform control related to a part of or the entire detaching processing described with reference to FIG. 3 by executing a control program recorded in a computer-readable recording medium. The control device 11 can perform control related to a part of or the entire detaching processing by, for example, sequence control, feedback control, or a combination of sequence control and feedback control.

The present disclosure can also be implemented by processing in which a program for implementing one or more functions of the embodiments is supplied to a system or a device via a network or a storage medium, and one or more processors in a computer of the system or the device read and execute the program. The present disclosure can also be implemented by a circuit (for example, an application specific integrated circuit (ASIC)) that implements one or more functions.

### OTHER EMBODIMENTS

Embodiment(s) of the present disclosure can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present disclosure has been described with reference to embodiments, it is to be understood that the present disclosure is not limited to the disclosed embodiments. The scope of the following claims is to be accorded the broadest interpretation so as to encompass all such modifications and equivalent structures and functions.

This application claims the benefit of Japanese Patent Application No. 2025-009481, filed Jan. 22, 2025, which is hereby incorporated by reference herein in its entirety.

What is claimed is:

1. A cell extraction method comprising the steps of:
injecting a first liquid containing a detachment reagent into a culture vessel in which a first cell and a second cell coexist to make an adhesion strength of the first cell lower than an adhesion strength of the second cell;
applying, by a first detachment mechanism, a first physical force to a content of the culture vessel via the first liquid to detach at least a part of the first cell and discharging from the culture vessel the first liquid containing the first cell detached;
supplying a second liquid to the culture vessel and applying, by a second detachment mechanism, a second physical force larger than the first physical force to the content of the culture vessel via the second liquid to detach the second cell present in the culture vessel; and
collecting from the culture vessel, by a collection mechanism, the second liquid containing the second cell detached.

2. The cell extraction method according to claim 1, wherein when applying a first physical force, a posture of the culture vessel is changed, or the culture vessel is rocked.

3. The cell extraction method according to claim 1, wherein when applying a second physical force, the culture vessel is rocked.

4. The cell extraction method according to claim 1, wherein an ejection mechanism included in the second detachment mechanism ejects the second liquid to the content of the culture vessel.

5. The cell extraction method according to claim 4, wherein the ejection mechanism includes a spray nozzle that ejects droplets of the second liquid.

6. The cell extraction method according to claim 1, wherein a stirring mechanism included in the second detachment mechanism causes the second liquid to flow in the culture vessel.

7. The cell extraction method according to claim 1, wherein 70% or more of the first cells present in the culture vessel prior to injecting the first liquid are detached.

8. The cell extraction method according to claim 1, further comprising, after discharging the first liquid from the culture vessel and before supplying the second liquid, injecting a washing liquid from a washing unit into the culture vessel to wash the content and then discharging the washing liquid from the culture vessel.

9. The cell extraction method according to claim 8, wherein 70% or more of the first cells present in the culture vessel prior to injecting the first liquid are discharged from an inside of the culture vessel with the washing liquid.

10. The cell extraction method according to claim 8, wherein 70% or more of the second cells present in the culture vessel prior to injecting the second liquid remain inside the culture vessel after injecting a washing liquid from a washing unit into the culture vessel to wash the content and then discharging the washing liquid from the culture vessel.

11. The cell extraction method according to claim 8, wherein 90% or more of the second cells present in the culture vessel after discharging the washing liquid are detached.

12. The cell extraction method according to claim 1, wherein
the first liquid is a solution containing a proteolytic enzyme and/or a chelating agent, and
the second liquid is physiological saline or a liquid culture medium.

13. The cell extraction method according to claim 8, wherein the washing liquid contains any one of physiological saline, a liquid culture medium, and a cryopreservation liquid.

14. The cell extraction method according to claim 1, wherein
the first cell is a partially reprogrammed cell in which a reprogramming factor has been introduced into a mononuclear cell, and
the second cell is an iPS cell in which a reprogramming factor has been introduced into a mononuclear cell and of which reprogramming has been completed.

15. A subculture method comprising:
subculturing the second cell contained in the second liquid collected in the collection step of the cell extraction method according to claim 1.

16. A cell extraction system comprising:
a first detachment mechanism configured to detach at least a part of a first cell from a culture vessel, wherein contents of the culture vessel comprise the first cell and a second cell which has a higher adhesion strength than the first cell which coexist in the culture vessel, by applying a first physical force to the contents of the culture vessel via a first liquid after the first and second cells are immersed in the first liquid containing a detachment reagent;
a second detachment mechanism configured to detach the second cell present in the culture vessel by applying a second physical force larger than the first physical force to the content of the culture vessel via a second liquid injected into the culture vessel after the first liquid containing the first cell detached is discharged from the culture vessel; and
a collection mechanism configured to collect from the culture vessel the second liquid containing the second cell detached.

17. The cell extraction system according to claim 16, further comprising a drive mechanism configured to change a posture of the culture vessel or rock the culture vessel.

18. The cell extraction system according to claim 16, wherein the second detachment mechanism includes an ejection mechanism configured to eject the second liquid to the content of the culture vessel.

19. The cell extraction system according to claim 18, wherein the ejection mechanism includes a spray nozzle configured to eject droplets of the second liquid.

20. The cell extraction system according to claim 16, further comprising a first injection unit configured to inject the first liquid.
